SEQUENCE LISTING (24 sequences)

SEQ ID NO: 1 - DNA, 29 bp, Artificial sequence
  Primer for T7 RNA polymerase gene
ggcctgaataggtacgaattcctaactgg

SEQ ID NO: 2 - DNA, 29 bp, Artificial sequence
  Primer for T7 RNA polymerase gene
tatagtgagtcgtatggatccggcgttac

SEQ ID NO: 3 - DNA, 26 bp, Artificial sequence
  Primer for T7 origin of replication
gatgttcctcggtgaattccgcttac

SEQ ID NO: 4 - DNA, 29 bp, Artificial sequence
  Primer for T7 origin of replication
ggtggtagaaggtaccagtatcaatcagg

SEQ ID NO: 5 - DNA, 35 bp, Artificial sequence
  Primer for T7 ssDNA binding protein gene
gaaacctaaaggaggaattcattatggctaagaag

SEQ ID NO: 6 - DNA, 28 bp, Artificial sequence
  Primer for T7 ssDNA binding protein gene
gcaccacacctgcccggatcctttattg

SEQ ID NO: 7 - DNA, 29 bp, Artificial sequence
  Primer for T7 helicase/primase gene
gggtaaacagcataagcttcgtagtagag

SEQ ID NO: 8 - DNA, 30 bp, Artificial sequence
  Primer for T7 helicase/primase gene
cctttagtgagtcatatgagaatgggactc

SEQ ID NO: 9 - DNA, 25 bp, Artificial sequence
  Primer for T7 DNA polymerase gene
tcaataggagaattcaatatgatcg

SEQ ID NO: 10 - DNA, 25 bp, Artificial sequence
  Primer for T7 DNA polymerase gene
ctttggtaagcttgtaggctactag

SEQ ID NO: 11 - DNA, 27 bp, Artificial sequence
  Primers for confirmation of proper insertion in tauA and tauB
caaatacgcggcttaaaacatattcgc

SEQ ID NO: 12 - DNA, 27 bp, Artificial sequence
  Primers for confirmation of proper insertion in tauA and tauB
aggggagcagacaatcatggcaatttc

SEQ ID NO: 13 - DNA, 27 bp, Artificial sequence
  Primers for confirmation of proper insertion in tauC
ctaaaagaaaggcgataatcgcaatca

SEQ ID NO: 14 - DNA, 24 bp, Artificial sequence
  Primers for confirmation of proper insertion in tauC
ctctggcaggagacgggcaagcag

SEQ ID NO: 15 - DNA, 27 bp, Artificial sequence
  Primer for Asp5Ala/Glu7Ala substitution in T7 DNA polymerase
gagggcgttagcagcgatagcagaaac

SEQ ID NO: 16 - DNA, 27 bp, Artificial sequence
  Primer for Asp5Ala/Glu7Ala substitution in T7 DNA polymerase
gtttctgctatcgctgctaacgccctc

SEQ ID NO: 17 - DNA, 30 bp, Artificial sequence
  Primer for Glu480Asp substitution in T7 DNA polymerase
cgcatccggtcttgatctacgctgcttggc

SEQ ID NO: 18 - DNA, 30 bp, Artificial sequence
  Primer for Glu480Asp substitution in T7 DNA polymerase
gccaagcagcgtagatcaagaccggatgcg

SEQ ID NO: 19 - DNA, 29 bp, Artificial sequence
  Primer for Tyr530Phe substitution in T7 DNA polymerase
ctatgggttcctctttggtgctggtgatg

SEQ ID NO: 20 - DNA, 29 bp, Artificial sequence
  Primer for Tyr530Phe substitution in T7 DNA polymerase
catcaccagcaccaaagaggaacccatag

SEQ ID NO: 21 - DNA, 28 bp, Artificial sequence
  Primer for Tyr100Pro substitution in tetracycline efflux pump
cctgtggattctccccgccggacgcatc

SEQ ID NO: 22 - DNA, 28 bp, Artificial sequence
  Primer for Tyr100Pro substitution in tetracycline efflux pump
gatgcgtccggcggggagaatccacagg

SEQ ID NO: 23 - DNA, 27 bp, Artificial sequence
  Primer for amplification of beta-lactamase from pUC18
ctacggggtctgaagcttagtggaacg

SEQ ID NO: 24 - DNA, 28 bp, Artificial sequence
  Primer for amplification of beta-lactamase from pUC18
ctgctcccgtgatcagcttacagacaag

(19) United States
(12) Patent Application Publication
(10) Pub. No.: US 2009/0269803 A1
(43) Pub. Date: Oct. 29, 2009

(54) In Vivo Generation of Dna, Rna, Peptide, and Protein Libraries

(75) Inventors: Andreas Meyer, Frauenfeld (CH); Eric Eichhorn, Strasbourg (CH)

Correspondence Address: WENDEROTH, LIND & PONACK, L.L.P., 1030 15th Street, N.W.,, Suite 400 East, Washington, DC, 20005-1503, US

(73) Assignee: Andreas Meyer, Frauenfeld (CH)

(21) Appl. No.: 11/992,314

(22) PCT Filed: Sep. 20, 2006
(86) PCT No.: PCT/EP2006/066535

§ 371 Date: Mar. 20, 2008

(30) Foreign Application Priority Data
Sep. 21, 2005 (EP) 05108694.0

(51) Int. Cl. C12P 21/00; C40B 50/06; C40B 30/06

(52) U.S. Cl. 435/69.1; 506/26; 506/10

(57) ABSTRACT

The present invention relates to a method for the in vivo generation of DNA, RNA, peptide and protein libraries by means of a genetic element harboring a viral or phage origin of replication that is independently reproduced by a viral or phage error-prone polymerase not physically linked to the genetic element within a host cell furthermore containing viral or phage auxiliary nucleotide sequences and proteins that are required for replication of the viral or phage genetic element. The nucleotide or nucleotides of interest to be diversified are introduced into the genetic element and physically linked to the viral or phage origin of replication.

### FIELD OF INVENTION

The present invention relates to a method for the generation of DNA, RNA, peptide, and protein libraries by mutagenesis within a living cell. The invention furthermore relates to the selection, production, and application of variants prepared by this method.

### BACKGROUND OF THE INVENTION

Biopolymers, such as DNA, RNA, peptides, and proteins, are used in a variety of biotechnological applications. Proteins and peptides are e.g. used in medicine as therapeutics (e.g. antibodies, vaccines, interferons, interleukins, soluble receptors, hormones, enzymes), in industry as catalysts, in households as part of detergents or cosmetics, or in nutrition as food/feed additives. The proteins used in these applications usually derive from natural sources, but may have been adapted to their use e.g. by substitution of amino acids of the original sequence and/or by other modifications (polyethylene glycol attachment, immobilization, cross-linking, etc.).

Traditionally, modification of amino acid sequences is done by site directed mutagenesis of the DNA encoding the corresponding protein. These alterations are accomplished based on elucidated sequence-structure-function relationships. As a result, this approach is only feasible for molecules, for which this detailed information is available.

Other methods of changing the amino acid sequence of a protein are based on the Darwinian principle of evolution, namely random diversification and subsequent selection (see e.g. Steipe, B., Curr. Top. Microbiol. Immunol. 243:55-86, 1999). Due to this principle, such approaches are also called ‘directed evolution’ experiments. In a first step, diversification of the gene encoding the protein of interest is randomly done to generate a DNA library. Next, from this DNA library the corresponding proteins are synthesized. Finally, the protein variants are subjected to a screening procedure and the variants with the desired properties are selected.

For the generation of diversity several procedures have been developed. Classical examples are the mutagenesis of entire organisms by radiation (UV, X-ray) or by chemical mutagens (ethyl methanesulfonate, hydroxylamine, etc.). Also mutator strains, which have low DNA replication fidelity, can be used for in vivo mutagenesis. These methods have the disadvantage that diversification does not only take place on the particular DNA of interest, but on the entire genome of the host. This results in loss of fitness of the host strain (Funchain, P., et al., Genetics 154:959-970, 2000), since vital genes may be destroyed, and, therefore, in low diversity. Thus, the advantage of being able to create diversity on the DNA level and simultaneously synthesize the corresponding proteins is nullified.

Targeted mutagenesis of the DNA coding for the protein of interest can efficiently be done in vitro. To this end, e.g. error-prone polymerase chain reaction (PCR) (Beckman, R. A., et al., Biochemistry 24:5810-5817, 1985) or DNA shuffling (Stemmer, W. P. C., Nature 370:389-391, 1994) can be used. Synthesis of the corresponding protein variants can subsequently be done in vivo or in vitro. In vivo synthesis requires the cloning of the DNA into an expression vector and the introduction of the construct into a living cell. These are two highly inefficient processes that drastically lower the diversity of the library (Dower, W. J., and Cwirla, S. E. in Chang, D. C., et al., (ed.), Guide to Electroporation and Electrofusion. Academic Press, San Diego, 1992). In vitro protein synthesis demands stringently defined conditions, such as low temperature and distinct salt concentrations, and is limited by correct folding of the products. As a result, the method is suitable only for specific applications. Regardless of the method of protein synthesis, such approaches require iterative switching between diversification and synthesis of the proteins, which is a troublesome and work intensive process.

Efforts have already been made to develop methods that enable the generation of diversity on a specific DNA segment within a living cell. WO 97/025410 (and corresponding U.S. Pat. No. 6,500,644) describes the use of a genetic element that is replicated by an error-prone DNA polymerase. Thereby, the origin of replication is connected to a polynucleotide of interest and, optionally, to a polynucleotide encoding the error-prone polymerase.

However, the properties of the genetic element and the principle of replication of this element differ strongly from the invention described here. In WO 97/025410, the same proteins are involved in the replication of the genetic element as well as of the host chromosome, and therefore base substitutions are inserted into both types of molecules. The minimization of mutations on the chromosome requires that some elements in the replication system may be temporally “switched” off, fully or partially, thereby stopping or greatly slowing down the replication of the chromosome, while replication of the genetic element is continued. As a consequence, diversification cannot be coupled to growth-selection. In contrast, the use of virus or phage derived genetic elements, as described in the present invention, allows the concomitant diversification and synthesis of proteins in growing cells, since the host chromosome is replicated by host enzymes, while the genetic element is replicated by virus or phage proteins. Thus, this fact enables direct coupling of diversification and selection and offers a major advantage over the prior art system, since progressive evolution is possible.

In WO 97/025410, also the use of a phagemid as a genetic element is envisaged. However, the phage origin of replication is explicitly used in order to couple generation of variants to a display system by filamentous phage, and not for error-prone replication of the genetic element. The gene of interest is fused to a phage coat protein, and infection with a helper phage is required. In addition, the application of entire bacteriophages containing error-prone DNA polymerases is considered. Nevertheless, such systems are not stable, since the error-prone DNA polymerase and the origin of replication are physically linked, which leads to modification of the gene of interest as well as to modification of the gene encoding the DNA polymerase and of other phage genes. In contrast to WO 97/025410, in the invention described herein the error-prone polymerase is not physically linked to the independently replicating genetic element. Furthermore, it does not involve the assembly of a functional phage with the optional display of the variant proteins on its surface. As a result, the present invention clearly differs from the prior art use of a bacteriophage containing an error-prone polymerase.

Loeb and coworkers (Camps, M., et al., PNAS 100:9727-9732, 2003; Shinkai, A., and Loeb, L. A., J. Biol. Chem. 276:46759-46764, 2001) used a system as described in WO 97/025410 for in vivo mutagenesis with an error-prone Escherichia coli DNA polymerase I. Since this polymerase is also involved in the replication of the host chromosome, mutations were introduced into the genome of the host cell. As mentioned above, this leads to loss of fitness of the host strain. Furthermore, although growth of the cells is minimized by steady state cultivation, residual DNA polymerase III is active in replication of the genetic element. As a consequence, mutations are accumulated around the origin of replication, where the DNA polymerase I initiates replication.

Directed evolution has proven to be a valuable tool for the design of biopolymers with specific properties. However, traditional approaches have limitations, such as e.g. low diversity and laborious experimental set-ups that include repetitive switching between diversification, expression, screening, and selection. Newer in vivo approaches have the disadvantage that mutations are also introduced into the chromosome of the host, which leads to loss of fitness. Furthermore, diversification cannot be coupled to selection, since growth of the host cells has to be minimized during diversification. As a result, progressive evolution is not possible. A method that allows the generation of diversity on a specific DNA segment, the concomitant synthesis of the corresponding proteins, and the simultaneous selection of improved variants could eliminate many constraints of current technologies, and is necessary to advance random protein design.

### SUMMARY OF THE INVENTION

The present invention relates to a method for the in vivo generation of DNA, RNA, peptide, and protein libraries by means of a genetic element that is independently reproduced by an error-prone polymerase within a host cell. Independent replication is achieved by using virus and/or bacteriophage related elements on the genetic element to be diversified itself and/or in the host cell.

More specifically, the invention comprises a method for the in vivo generation of a library of variants of polynucleotides comprising culturing a host cell wherein the host cell

i) contains a genetic element harboring a viral or phage origin of replication,
ii) harbors a viral or phage error-prone polymerase that is involved in replication of said genetic element (i), but which is not physically linked to said genetic element (i),
iii) harbors viral or phage auxiliary nucleotide sequences and proteins that are required for replication of said genetic element (i),
iv) contains a nucleotide sequence or several nucleotide sequences of interest that are physically linked to said viral or phage origin of replication (i),
v) replicates its genome independently of said genetic element (i).

The invention further relates to the use of a virus or phage derived independently replicating element in directed evolution experiments. In particular, the invention relates to the generation of a polynucleotide library by introducing a nucleotide sequence of interest into the genetic element, growing the host cells harboring said genetic element, thereby introducing diversity into the nucleotide sequence of interest, performing screening and/or selection of cells harboring a desired variant with improved properties, and isolating the corresponding polynucleotide. If desired, this cycle is repeated, entirely or in part, until a polynucleotide with the desired properties is obtained.

Thus, the invention furthermore relates to a method for the generation of polynucleotides with desired properties or polynucleotides encoding proteins with desired properties, wherein

i) a library of nucleotide sequence variants is constructed by culturing a host cell as described hereinbefore,
ii) said library (i) is screened and selected for host cells producing variants with desired properties,
iii) said selected host cells (ii) are isolated,
iv) the variant nucleotide sequences of interest on the genetic elements of said isolated host cells (iii) are isolated and characterized.

The invention further relates to the manufacture of peptides or proteins wherein a variant nucleotide sequence with desired properties is generated and isolated by the method as described hereinbefore, and then used for the production of encoded peptides or proteins in a suitable host cell.

The invention further relates to the use of such peptides or proteins, in particular as a therapeutic, catalyst, detergent, cosmetic or feed additive.

### DETAILED DESCRIPTION OF THE INVENTION

A “polynucleotide” (or “nucleotide sequence”) is a DNA or RNA obtainable by linking several nucleotides.

A “protein” (or “peptide”) is obtainable by linking several amino acids, e.g. α-amino acids, and may be further processed, e.g. by glycosylation.

A “polymerase” is an enzyme, such as e.g. a DNA polymerase, an RNA polymerase, or a reverse transcriptase that catalyzes the formation of polynucleotides of DNA or RNA using an existing strand of DNA or RNA as a template.

An “error-prone polymerase” is a polymerase that incorporates mistakes, e.g. wrong nucleotides, or causes deletions or insertions of one or several nucleotides, during replication of DNA or RNA at a higher rate than the polymerase normally used for this purpose. “Fidelity” describes the accuracy of replication. Accordingly an error-prone polymerase has low fidelity, e.g. has a mutation rate equal or higher than 10⁻⁶ mutations per nucleotide per replication cycle.

A “virus” is a small particle that infects cells in biological organisms. The term “virus” usually refers to those particles that infect eukaryotes (multi-cell organisms and many single-cell organisms), whilst the term “bacteriophage” or “phage” is used to describe those particles infecting prokaryotes (bacteria and bacteria-like organisms). A “virion” is a single virus particle, complete with coat. Of “viral or phage origin” means derived from a virus or bacteriophage.

The “genome” is the whole hereditary information of an organism that is encoded in the DNA or, for some viruses, in the RNA. In the context of the description of this invention the term “genome” does not include the information encoded on the “independently replicating element”.

In the context of this invention, an “independently replicating (genetic) element” is an element consisting of a polynucleotide, either DNA or RNA, that is not replicated by the same enzymes as the chromosomes of the host cell. A “mutagenizing vector” is an independently replicating element that is replicated at low fidelity.

“Rational design” is the engineering of DNA, RNA, peptides, or proteins based on elucidated sequence-structure-function relationships.

“Random design” is the engineering of DNA, RNA, peptides, or proteins by methods that are based on the Darwinian principle of evolution, i.e. random diversification and selection. Experiments applying random design are therefore also called “directed evolution” experiments.

“Progressive evolution” is a sub-form of directed evolution, in which diversification is coupled to selection (e.g. growth), i.e. that beneficially modified variants are further diversified at a higher rate than other members of the library. As a consequence they are enriched over time.

An “origin of replication” is a specific DNA sequence at which DNA replication is initiated. DNA replication may proceed from this point bidirectionally or unidirectionally.

A “gene of interest” is a DNA segment with specific properties, typically coding for the “protein of interest”.

The invention relates to a method for the in vivo generation of a library of variants of polynucleotides comprising culturing a host cell wherein the host cell

i) contains a genetic element harboring a viral or phage origin of replication,
ii) harbors a viral or phage error-prone polymerase that is involved in replication of said genetic element (i), but which is not physically linked to said genetic element (i),
iii) harbors viral or phage auxiliary sequences and proteins that are required for replication of said genetic element (i),
iv) contains a nucleotide sequence or several nucleotide sequences of interest that are physically linked to said viral or phage origin of replication (i),
v) replicates its genome independently of said genetic element (i).

The invention comprises a method to generate a library of variants of polynucleotides, in particular DNA libraries in vivo, and herewith to obtain random variants of DNA, RNA, peptides, or proteins with altered properties. The method involves the use of a genetic element that harbors the nucleotide sequence(s) of interest, which is independently reproduced from the chromosome of the host cell.

Independent replication of the genetic element is achieved by using elements involved in the replication of viruses or bacteriophages, such as e.g. recognition sequences and genes, which are introduced into the host cell.

The DNA, RNA, peptides, or proteins obtained by this method may have altered properties such as, for example, altered physical, chemical, biochemical or biological properties. Particular molecules with changed properties can be, but are not limited to, antibodies with enhanced affinity, RNA with increased half-life time, enzymes with higher activity in organic solvents, receptor ligands showing superior specificity or trigger a higher response, or biocatalysts having a different substrate spectrum.

Viruses and bacteriophages are entities whose genome comprises polynucleotides, either DNA or RNA, which reproduce inside living cells. They are obligate intracellular parasites and lack the enzymes required for energy production. The genome of viruses and bacteriophages is usually replicated at higher mutation rates compared to replication of the genome of the host cell. As a consequence, their offspring evolve rapidly, which is of advantage for evading common defense mechanisms of the cell. The reason for the increased mutagenesis of the viral genetic information is the lower fidelity of their DNA polymerases, reverse transcriptases or RNA polymerases.

The genome of viruses and bacteriophages contains all the information necessary to produce their progeny within the host cell. However, replication in the cell depends on the virus type. Some DNA viruses and bacteriophages are exclusively replicated by host enzymes; their genome encodes only structural proteins and enzymes required for the release of newly assembled particles. The genome of other DNA viruses and bacteriophages encodes in addition proteins that are involved in initiation of DNA replication and DNA replication. Similarly, RNA viruses use different procedures for replication. One possibility is the replication of the viral genome by RNA dependent RNA polymerases (RNA replicases). Newly synthesized RNA can be directly packed to assemble new virions. Other genomes of RNA viruses encode a reverse transcriptase that transcribes the RNA into DNA. Host RNA polymerases subsequently produce RNA molecules that can be packed into virus particles.

An important aspect of the invention is the construction of a genetic element that is reproduced by means of virus or bacteriophage proteins. For example, elements from a bacteriophage may be used to construct the independently reproducing genetic element. The elements that are involved in maturation, packaging, and export of the virion are deleted from the genome of the bacteriophage. The trans-acting factors are excised from the reduced bacteriophage genome and separately introduced into the host cell. The residual cis-acting factors are used as a backbone for the construction of the independently reproducing genetic element, and can be complemented with, for example, genetic markers and the gene encoding the protein of interest. Since replication of viruses and bacteriophages usually proceeds with increased mutagenesis rates, it also allows the introduction of diversity into the genetic element. In this respect, the invention explicitly includes the engineering of virus or bacteriophage sequences and genes, for example, by rational or random design, to lower the fidelity of the replication of the genetic element.

The invention relates to the use of elements that are involved in virus and bacteriophage replication for the application in directed evolution experiments. Directed evolution is a term that includes methods based on the Darwinian principle of evolution, namely the random generation of diversity and selection. For the directed evolution of a protein, the first experimental step is usually the generation of diversity at the level of DNA or RNA.

Thus, the invention furthermore relates to a method for the generation of polynucleotides with desired properties or polynucleotides encoding proteins with desired properties, wherein

i) a library of nucleotide sequence variants is constructed by culturing a host cell as described hereinbefore,
ii) said library (i) is screened and selected for host cells producing variants with desired properties,
iii) said selected host cells (ii) are isolated,
iv) the variant nucleotide sequences of interest on the genetic elements of said isolated host cells (iii) are isolated are characterized

Screening of polynucleotides may include the synthesis of the proteins from these nucleic acids, and results in the construction of a protein library. Subsequently, this library is screened for members with desired properties, and the corresponding DNA or RNA molecules encoding the selected variants are isolated. If required, another round of diversification and selection is performed. This round of diversification and selection may be repeated for one or more times, e.g. one to ten times, in particular one to three times.

### Construction of Independently Reproducing Elements

The virus or bacteriophage elements required for the construction of an independently replicating element are assembled using methods of genetic engineering well known in the art. This is achieved based on knowledge how the replication process of a virus or a bacteriophage and its host proceeds. The genetic element harbors at least one virus or bacteriophage related sequence from which, for example, replication is initiated. Virus or bacteriophage related sequences include, but are not limited to, sequences directly isolated from viruses or bacteriophages, sequences that were amplified from viruses or bacteriophages, sequences that were designed based on original sequences of viruses or bacteriophages, and also sequences on which virus or bacteriophage derived proteins interact and e.g. initiate replication. The sequences may also be changed by rational or random design to improve their function in the independently reproducing element. Such alterations may include, for example, changing the nucleotide sequence or adding and/or deleting specific nucleotides.

The independently replicating genetic element may optionally also encode a genetic marker, e.g. a gene conferring resistance towards an antibiotic or a gene coding for a metabolic enzyme. The marker enables host cells containing the independently replicating genetic element to proliferate under specific conditions, while cells without are not able to multiply. As a result, the final library only contains cells harboring variants of the nucleotide sequence of interest. If direct selection for improved nucleotide sequences or proteins derived from improved nucleotide sequences can be performed, the introduction of a marker is not required.

The independently replicating genetic element may also harbor recognition sequences for restriction enzymes. Such sequences enable the easy insertion of one or more nucleotide sequences of interest. If desired, a heterologous promoter can be inserted in front of the above-mentioned restriction sites. In doing so, controlled high-level expression of the inserted nucleotide sequence of interest is possible.

Optionally, the genetic element may in addition contain a plasmid origin of replication. This fact allows, besides virus or bacteriophage based replication, also the replication of the genetic element by the host cell. Optimally, replication from the plasmid origin is tightly controlled. This is e.g. achieved by using a pSC101 origin of replication and providing the RepA protein in trans, which is essential for replication (Xia, G., et al., J. Bacteriol. 175:4165-4175, 1993). For replication from the virus or bacteriophage origin of replication, the corresponding viral or phage genes are expressed. To replicate the genetic element by means of the host replication machinery, expression of the repA gene is induced.

Suitable viruses for the construction of the genetic element are double stranded DNA (dsDNA) viruses, single stranded DNA (ssDNA) viruses, dsRNA viruses, (+)ssRNA viruses (positive-sense), (−)ssRNA viruses (negative-sense), reverse transcribing RNA viruses, reverse transcribing DNA viruses, naked RNA viruses, or subviral agents. Preferred dsDNA viruses are from the order caudovirales. Preferred caudovirales are from the families Podoviridae and Myoviridae. Preferred Podoviridae are from the genera “T7-like viruses”, “P22-like viruses”, and “(φ29-like viruses”. Preferred “T7-like virus” is Enterobacteria phage T7. Preferred “P22-like virus” is Enterobacteria phage P22. Preferred “(φ29-like viruses” are Bacillus phage φ29, Bacillus phage B103, and Bacillus phage GA 1. Preferred Myoviridae are from the genus “T4-like viruses”. Preferred “T4-like viruses” are Enterobacteria phage T4, Enterobacteria phage RB69, and Enterobacteria phage RB49. Other preferred dsDNA viruses are from the families of Herpesviridae and Adenoviridae. Preferred reverse transcribing DNA viruses belong to the families Hepadnaviridae and Caulimoviridae. Preferred Hepadnaviridae are from the genus Orthohepadnavirus. Preferred Orthohepadnavirus is Hepatitis B virus. Preferred Caulimoviridae are from the family Caulimovirus. Preferred Caulimovirus is Cauliflower mosaic virus. Preferred dsRNA viruses belong to the families of Cystoviridae and Totiviridae. Preferred Cystoviridae are from the genus Cystovirus. Preferred Cystovirus is Pseudomonas phage φ6. Preferred Totiviridae are from the genus Totivirus. Preferred species from Totivirus are Saccharomyces cerevisiae virus L-A and Saccharomyces cerevisiae virus L-BC. Preferred (+)ssRNA viruses belong to the family Leviviridae. Preferred Leviviridae are from the genus Levivirus. Preferred Levivirus is Enterobacteria phage MS2. Other preferred (+)ssRNA viruses are from the order nidovirales. Preferred nidovirales belong to the family Togaviridae. Preferred Togaviridae are from the genus Alphavirus. Preferred Alphavirus is Sindbis virus. Preferred (−)ssRNA viruses belong to the order mononegavirales. Preferred mononegavirales belong to the family Rhabdoviridae. Preferred Rhabdoviridae are from the genus Vesiculovirus. Preferred Vesiculovirus is Vesicular stomatitis virus. Preferred reverse transcribing RNA viruses belong to the families Pseudoviridae, Metaviridae, and Retroviridae. Preferred Pseudoviridae are from the genus Pseudovirus and Hemivirus. Preferred Pseudovirus is Saccharomyces cerevisiae Ty1 virus. Preferred Hemivirus is Saccharomyces cerevisiae Ty5 virus. Preferred Metaviridae are from the genus Metavirus. Preferred Metavirus is Saccharomyces cerevisiae Ty3 virus. Preferred Retroviridae are from the genus Lentivirus. Preferred Lentivirus is Human immunodeficiency virus 1. Preferred naked RNA viruses belong to the family Narnaviridae. Preferred Narnaviridae are from the genus Narnavirus. Preferred Narnavirus is Saccharomyces cerevisiae 23SRNA narnavirus. Preferred subviral agents are satellites. Preferred satellites are satellite polynucleotides. Preferred satellite polynucleotides are double-stranded satellite RNAs. Preferred double-stranded satellite RNA is the satellite of Saccharomyces cerevisiae M virus.

### Construction of Host Strains

Introducing genes from viruses or bacteriophages, whose expression allows the independent replication of the genetic element, is used for the construction of the host strains. Typically, at least a gene encoding a polymerase, such as a DNA polymerase, an RNA polymerase, or a reverse transcriptase, will be inserted into the host strain. The polymerase may contain the original wild-type sequence or may be modified by protein engineering.

For replication of the virus or bacteriophage derived element it is necessary to introduce auxiliary proteins. Such elements include, but are not limited to, genes encoding primases, helicases, helicase loaders, single-stranded DNA (ssDNA) binding proteins, double-stranded DNA (dsDNA) binding proteins, RNA polymerases, clamps, sliding clamps, clamp loaders, initiator proteins, origin binding proteins, polymerase accessory proteins, replisome organizer proteins, DNA ligases, RNases, topoisomerases, exonucleases, or endonucleases. The kind of auxiliary proteins inserted depends on the virus or bacteriophage from which the genetic element derives. They are selected based on knowledge of the mechanism of replication of the virus or phage. For example, for bacteriophage T7 derived genetic elements, in addition to the T7 DNA polymerase also the T7 RNA polymerase, the T7 ssDNA binding protein, and the T7 helicase/primase protein may be introduced into the host cell. For bacteriophage T4 derived genetic elements, in addition to the T4 DNA polymerase also T4 ssDNA binding protein, T4 helicase, T4 clamp, T4 clamp loader, T4 helicase loader, T4 topoisomerases, T4 RNase H, and T4 DNA ligase may be introduced into the host cell. In an analogous manner, for herpes simplex virus (HSV) derived genetic elements, in addition to the HSV DNA polymerase also HSV accessory protein, HSV origin binding protein, HSV helicase/primase complex, and HSV ssDNA binding protein may be introduced into the host cell. Thereby, it is not a requirement that the auxiliary proteins derive from the same virus or bacteriophage as the polymerase. Also heterologous proteins can be used, if they are able to substitute for a specific protein function.

Methods for expressing recombinant genes in different cells are well known in the art. The corresponding DNA sequences can e.g. be inserted on plasmids, on cosmids, on artificial chromosomes, or inserted into the host chromosome by homologous recombination, by viral or phage insertion, or by transposon mutagenesis. The expression of the introduced genes may be controlled by their own regulatory sequences, but also from heterologous promoters. Alternatively, the polymerase and/or the auxiliary proteins can also be provided by a virus or phage. Such helper viruses and phages may optionally be deficient in specific functions, such as host lysis or particle formation.

Introduction of the Genetic Element into Host Cells

In general, viruses and bacteriophages have high host specificity. This specificity is usually determined by the mechanism for entering the cell. The preferred host cells are the natural hosts of the virus or bacteriophage. As a consequence, for genetic elements constructed with elements from coliphages, the preferred host is Escherichia coli, with elements from Hepatitis virus, the preferred cells are mammalian cells, with elements from φ29, the preferred host is Bacillus, and so forth. However, the invention also relates to the use of genetic elements in heterologous hosts. In this case, it may also be necessary to transfer host factors into the cells replicating the genetic element. For example, by using a genetic element derived from bacteriophage T7 also the host factor Escherichia coli thioredoxin may have to be transferred into the heterologous host.

Suitable host cells are from any taxonomic origin, including archaea, eubacteria, and eukaryota. Preferred host cells from eubacterial origin are proteobacteria and firmicutes. Preferred proteobacteria are γ-proteobacteria. Preferred γ-proteobacteria are enterobacteriales and pseudomonales. Preferred enterobacteriales belong to the family Enterobacteriaceae. Preferred Enterobacteriaceae are from the genus Escherichia. Preferred Escherichia species is Escherichia coli. Preferred pseudomonales belong to the family Pseudomonaceae. Preferred Pseudomonaceae are from the genus Pseudomonas. Preferred Pseudomonas species are Pseudomonas syringae and Pseudomonas putida. Preferred firmicutes are bacilli. Preferred bacilli are bacillales. Preferred bacillales belong to the family Bacillacea. Preferred Bacillaceae are from the genus Bacillus. Preferred Bacillus species is Bacillus subtilis. Preferred host cells from eukaryotic origin are ascomycote fungi. Preferred ascomycote fungi are the hemi-ascomycetous yeasts. Preferred hemi-ascomycetous yeasts belong to the family Saccharomycetaceae. Preferred Saccharomycetaceae are from the genera Saccharomyces and Pichia. Preferred Saccharomyces species is Saccharomyces cerevisiae. Preferred Pichia species is Pichia pastoris. Likewise preferred are host cells from eukaryotic origin, such as host cells from the phylum Chordata. Preferred chordates are mammals. Preferred mammalian cell lines are CHO, HeLa, SupT1, COS-7, NIH 3T3, and T47D cell lines.

The genetic elements are introduced into host cells by known methods in the field, such as physical (e.g. electroporation, injection, biolistics), chemical (e.g. DMSO, PEG, chloride, liposomal transfection), biological (e.g. phage, virus transduction), or other methods. A genetic marker introduced into the independently reproducing element allows for easy selection of transformed cells.

### Generation of Diversity

Replication of viruses and bacteriophages usually takes place at lower accuracy than the replication of the host cells, thus enabling to evade defense mechanisms of the cell. This low fidelity replication is used to generate diversity on the independently replicating genetic element.

It is an aspect of the invention to increase the diversity of the constructed library by lowering the replication fidelity of the independently replicating genetic element. This can be achieved, for example, by increasing the misincorporation rate of the polymerase, or by deleting existing proofreading activities. To do so, rational design by site-directed mutagenesis is e.g. used to change the nucleotide recognition domain of the enzyme, or to eliminate the exonuclease activity of the protein. It is evident that also random approaches can be applied for the design of polymerases with desired fidelity. A library of random polymerases can be constructed by conventional methods such as, for example, error-prone PCR or DNA shuffling. Placing an inactivated marker gene into the genetic element can be used for the selection of low fidelity polymerases. The genetic element and the polymerase variants are co-inserted into the host cell and, after growth, selection for a reconstituted marker gene is performed. Finally, the polymerases are isolated and characterized.

Lowering the fidelity of the replication of the genetic element does not necessarily require engineering of the polymerase. Another possibility is to alter auxiliary virus, bacteriophage, or host proteins that are involved in the replication process. This can be done exclusively or in addition to the modification of the polymerase.

It is an important aspect of the invention, that different polymerases are involved in the replication of the genetic element and the genome of the host cell. The viral or phage low fidelity polymerase replicates the genetic element, thus introducing diversity into the nucleotide sequence(s) of interest. In contrast, the genome of the host is replicated by the intrinsic host polymerase at natural accuracy, thus allowing the host cells to preserve their fitness.

Construction of a DNA, RNA, peptide, or protein library is achieved by growing host cells, which produce replication proteins with the desired fidelity, and which harbor the independently replicating genetic element containing the nucleotide sequence(s) of interest. As starting point, either a defined molecule can be used or a library that has been produced by any in vivo or in vitro diversification procedure known in the art, such as error-prone PCR or StEP recombination. Further diversity is generated during growth of the host cells, which results in the construction of the desired library. DNA libraries can directly be isolated from DNA virus or bacteriophage derived genetic elements. RNA, peptide, or protein libraries can be obtained from the same elements after induction of DNA expression and RNA translation. DNA libraries can be obtained from RNA based genetic elements by using reverse transcriptases, whereas RNA libraries can be directly obtained. Peptide or protein libraries can be obtained after translation of the RNA sequences.

### Screening and Selection

The DNA, RNA, peptide, or protein libraries obtained are subsequently screened or subjected to selection by methods well known in the art. By the screening process, members of the library with specific properties are identified, and can be isolated in a following step. Screening usually is a high-throughput process and, therefore, often performed automatically. For example, optical measurements such as monitoring absorbance or fluorescence are used to identify variants with desired properties. Subsequently, individual members are isolated by cell sorting (e.g. FACS).

A selection procedure combines the process of identification and isolation in a single step. For example, the desired property is coupled to the growth of the cell in such a way that only cells harboring variants with the desired feature are able to grow. This can be done e.g. by applying two-hybrid systems when screening for binding properties, by using knock out hosts for the engineering of metabolic proteins, or when proteins are evolved that yield resistance to environmental conditions, such as for example temperature or the presence of toxic substances. Another selection method, for which many techniques have been developed, is affinity selection. Thereby, the library is expressed on the surface of a cell or a phage, and affinity panning is performed on an immobilized binding partner.

It is an important aspect of the invention that diversification is done during growth of the host cell. As a result, selection procedures coupled to cell growth lead to a progressive optimization of already improved variants. For example, the evolution of an enzyme involved in the biosynthesis of a vital compound can be performed. Therefore, the wild-type gene on the genome of the host is inactivated e.g. by deletion using homologous recombination. A synthetic DNA library is then cloned into the independently reproducing genetic element and transformed into the prepared host. The cells are allowed to grow on medium supplying the vital compound for initial growth. During this stage diversity is generated on the genetic element and the concentration of the vital compound is decreased. After complete depletion, only cells that have acquired the ability to synthesize this compound will be able to grow. Moreover, cells with better enzymatic power will grow faster and consequently undergo further diversification. Over all seen, host cells with the best biosynthetic enzyme will be enriched and can be isolated. Such a process is referred to as progressive evolution.

Isolation of Sequence with Desired Properties

After selection of the cell containing the molecule with the desired properties, the corresponding polynucleotide is isolated by one of the methods well known in the art. For example, if the target molecule is a protein with specific properties, the DNA encoding the same is isolated. To this end, the genetic element harboring the gene can be isolated from the host cell, or the gene of interest can be amplified by PCR from isolated DNA or directly from cell lysates. The DNA is subsequently analyzed by standard methods and can be further used for additional modifications or synthesis of the protein of interest.

### Kits Suitable for the Invention

The invention furthermore relates to a kit that can be used for the construction of DNA, RNA, peptide, or protein libraries based on the method described herein. Such a kit comprises at least two out of the three components being

i) a genetic element harboring a viral or phage origin of replication as described hereinbefore, into which a nucleotide sequence or several nucleotide sequences of interest can be introduced,
ii) a nucleotide sequence encoding a viral or phage error-prone polymerase that is involved in replication of said genetic element (i), and
iii) one or several nucleotide sequences coding for auxiliary proteins that are required for replication of said genetic element (i).

A nucleotide sequence or several nucleotide sequences of interest can be introduced into a genetic element, if the genetic element contains e.g. suitable restriction sites to be cut by restriction enzymes in order to ligate the nucleotide sequence(s) with the genetic element in such a way that replication is enabled. In the kit, the genetic element can be provided as such or already cut for simplified insertion of the nucleotide sequence(s).

The nucleotide sequence encoding an error-prone polymerase that is involved in replication of said genetic element (i) may be provided either as such, in a host cell, or on an element of choice, e.g. on a plasmid, cosmid, or artificial chromosome. Examples of such nucleotide sequences are described hereinbefore.

A nucleotide sequence or several nucleotide sequences encoding auxiliary proteins required for replication of said genetic element (i) may be provided as such, in a host cell as described hereinbefore, or on an element of choice, e.g. on a plasmid, cosmid, or artificial chromosome.

Other components of the kit may be standard laboratory equipment, media to grow and replicate the host strain, the host strain itself, nucleotide sequences encoding suitable markers or being useful as markers, and the like.

### EXAMPLES

### Example 1

### Library Generation with Elements from Bacteriophage T7 and Reconstitution of a Tetracycline Efflux Pump

Bacteriophage T7 is a lytic E. coli phage containing a linear duplex DNA molecule with a size of approximately 40'000 base pairs. Most proteins required for replication are encoded on its genome, which enables bypassing the replication machinery of the host cell (Richardson, C. C., Cell 33:315-317, 1983).

Isolation of the Elements Required for Replication from the Bacteriophage T7

The elements required for the initiation of DNA replication and for the DNA replication itself are isolated from bacteriophage T7 DNA by polymerase chain reaction (PCR). The primary origin of replication is amplified using the oligonucleotides 5′-GATGTTCCTCGGTGAATTCCGCTTAC-3′ (SEQ ID NO: 3) and 5′-GGTGGTAGAAGGTACCAGTATCAATCAGG-3′ (SEQ ID NO: 4), which introduce an EcoRI restriction site at the 5′-end and a BamHI restriction site at the 3′-end of the amplified gene. The T7 RNA polymerase gene is amplified using the oligonucleotides 5′-GGCCTGAATAGGTACGAATTCCTAACTGG-3′ (SEQ ID NO: 1) and 5′-TATAGTGAGTCGTATGGATCCGGCGTTAC-3′ (SEQ ID NO: 2), which introduce an EcoRI restriction site at the 5′-end and a BamHI restriction site at the 3′-end of the amplified gene. The T7 single stranded DNA (ssDNA) binding protein gene is amplified using the oligonucleotides 5′-GAAACCTAAAGGAGGAATTCATTATGGCTAAGAA G-3′ (SEQ ID NO: 5) and 5′-GCACCACACCTGCCCGGATCCTTTATTG-3′ (SEQ ID NO: 6), which introduces an EcoRI restriction site at the 5′-end and a BamHI restriction site at the 3′-end of the amplified gene. The T7 helicase/primase gene is amplified using the oligonucleotides 5′-GGGTAAACAGCATAAGCTTCGTAGTAG AG-3′ (SEQ ID NO: 7) and 5′-CCTTTAGTGAGTCATATGAGAATGGGACTC-3′ (SEQ ID NO: 8), which introduce a HindIII restriction site at the 5′-end and an Ndel restriction site at the 3′-end of the amplified gene. The T7 DNA polymerase is amplified using the oligonucleotides 5′-TCAATAGGAGAATTCAATATGATCG-3′ (SEQ ID NO: 9) and 5′-CTTTGGTAAGCTTGTAGGCTACTAG-3′ (SEQ ID NO: 10), which introduce an EcoRI restriction site at the 5′-end and a HindIII restriction site at the 3′-end of the amplified gene.

### Cloning of the Genes Encoding the Proteins Involved in Bacteriophage T7 Replication

The genes encoding the proteins involved in the initiation of DNA replication and in DNA replication of bacteriophage T7 are cloned into plasmid pUC18 (Yanish-Perron, C., et al., Gene 33:103-109, 1985) using the restriction sites introduced by PCR. Sequencing is used to check the cloned genes for misincorporation of bases during amplification. Expression of the genes is done under the control of the tac promoter. Therefore, the T7 helicase/primase gene is excised as Sspl/EcoRI fragment from the above mentioned pUC18 derivative and ligated into Smal/EcoRI cut pKQV4 (Strauch, M. A., et al., EMBO J. 8:1615-1621, 1989). The T7 RNA polymerase and the T7 ssDNA binding protein are excised as EcoRI/HindIII fragments from the corresponding pUC18 derivatives and inserted into EcoRI/HindIII digested pKQV4.

### Construction of the Host Strain

For easier handling, the genes coding for bacteriophage T7 RNA polymerase, ssDNA binding protein, and helicase/primase are introduced into the E. coli JM101 chromosome by homologous recombination. To do so, they are transferred from the pKQV4 derivatives into the tauABCD operon of E. coli (van der Ploeg, J. R., et al., J. Bacteriol. 178:5438-5446, 1996) that was inserted as HindIII/Dral fragment into HindIII/Smal cut pUC18Notl (Herrero, M., et al., J. Bacteriol. 172:6557-6567, 1990). The genes encoding the replication proteins flanked by the tau sequence are subsequently cloned as Notl fragments into plasmid pKO3 (Link, A. J., et al., J. Bacteriol. 179:6228-6237, 1997). Homologous recombination is done as described by Link et al. (supra). The accurate insertion of the recombinant genes is confirmed by PCR using the oligonucleotides 5′-CAAATACGCGGCTTAAA ACATATTCGC-3′ (SEQ ID NO: 11) and 5′-AGGGGAGCAGACAATCATGGC AATTTC-3′ (SEQ ID NO: 12) for insertions in tauA and tauB, and with the oligonucleotides 5′-CTAAAAGAAAGGCGATAATCGCAATCA-3′ (SEQ ID NO: 13) and 5′-CTCTGGCAGGAGACGGGCAAGCAG-3′ (SEQ ID NO: 14) for insertions into tauC.

### Lowering the Fidelity of the Bacteriophage T7 DNA Polymerase

To achieve higher diversity in the produced DNA library, a bacteriophage T7 DNA polymerase with lowered fidelity is generated. As a first step, the exonuclease activity of the bacteriophage T7 DNA polymerase is eliminated; this results in lowering the fidelity by approximately a factor of 10 (Kunkel, T. A., et al., Proc. Natl. Acad. Sci. USA 91:6830-6834, 1994). To this end, site directed mutagenesis of residues Asp5 and Glu7 to Ala is carried out on a pUC18 plasmid derivative that contains the T7 DNA polymerase gene using the QuikChange site directed mutagenesis kit (Stratagene) and the oligonucleotides 5′-GAGGGCGTTAGCAGCGATAGCAGAAAC-3′ (SEQ ID NO: 15) and 5′-GTTTCTGCTATCGCTGCTAACGCCCTC-3′ (SEQ ID NO: 16). In a second step, amino acids involved in nucleotide recognition are mutated by the same method with oligonucleotides pairs 5′-CGCATCCGGTCTTGATCTACGCTGCTTGGC-3′ (SEQ ID NO: 17)/5′-GCCAAGCAGCGTAGATCAAGACCGGATGCG-3′ (SEQ ID NO: 18) for Glu480Asp substitution and 5′-CTATGGGTTCCTCTTTGGTGCTGGTGATG-3′ (SEQ ID NO: 19)/5′-CATCACCAGCACCAAAGAGGAACCCATAG-3′ (SEQ ID NO: 20) for Tyr530Phe substitution to further lower the fidelity of the bacteriophage T7 DNA polymerase (Donlin, M. J., and Johnson, K. A., Biochemistry 33:14908-14917, 1994).

### Construction of the Mutagenizing Vector

To construct the independently replicating genetic element that serves as mutagenizing vector, the PCR product containing the bacteriophage T7 origin of replication is digested with Kpnl and ligated into Kpnl digested plasmid pCK01, which is a pSC101 derivative. Before using in the directed evolution experiment, the gene encoding the RepA protein, responsible for replication of the plasmid by host enzymes, is eliminated from the genetic element.

### Reconstitution of an Inactivated Tetracycline Efflux Pump

The gene encoding the tetracycline efflux pump is cloned into the mutagenizing vector and inactivated by Tyr100Pro substitution (Brakmann, S., and Grzeszik, S., Chembiochem 2:212-219, 2001) using the QuikChange mutagenesis kit (Stratagene) with the oligonucleotides 5′-CCTGTGGATTCTCCCCGC CGGACGCATC-3′ (SEQ ID NO: 21) and 5′-GATGCGTCCGGCGGGGAGAATCCACAGG-3′ (SEQ ID NO: 22), according to the manufacturer's protocol. The error-prone bacteriophage T7 DNA polymerase on plasmid pUC18 is co-transformed with the genetic element harboring the gene encoding the inactive tetracycline pump into the constructed E. coli JM101 host strain. The cells are allowed to grow on LB medium, containing 200 μM IPTG for recombinant gene expression, and ampicillin and chloramphenicol as selection markers for the pUC18 derivative and the genetic element. After growing the culture to a density of approximately 0.3 g cell dry weight (CDW) per liter, tetracycline is added to a final concentration of 5 mg per liter. In doing so, only cells expressing a reactivated tetracycline efflux pump are allowed to grow further and are enriched in the culture. When the density reaches about 2 g CDW per liter aliquots are plated out on LB agar plates supplemented with IPTG, ampicillin, and tetracycline. Most colonies obtained after overnight incubation at 37° C. express a reconstituted tetracycline resistance gene, either by backmutation of Tyr100 or second site complementation, which is confirmed by sequencing.

### Example 2

### Directed Evolution of T7 DNA Polymerase Towards Low Fidelity

For different directed evolution experiments performed with the method described here, the use of polymerases with distinct fidelities may be envisaged. This example illustrates the selection of T7 DNA polymerase variants with low fidelity by use of the genetic element shown in Example 1.

### In Vitro Diversification of the T7 DNA Polymerase Gene

A library of T7 DNA polymerase variants is generated in vitro. To do so, the T7 DNA polymerase gene is amplified using in vitro manganese mutagenesis (Beckmann et al., supra). For the PCR reaction, a 100 μl volume containing 50 mM KCl, 10 mM Tris-HCl (pH 9), 6.5 mM MgCl₂, 0.1% Triton X-100, 10 μl DMSO, 0.5 mM MnCl₂, 1 mM dNTPs, 15 μM of primer 5′-TCAATAGGAGAATTCAATATGATCG-3′ (SEQ ID NO: 9), 15 μM of primer 5′-CTTTGGTAAGCTTGTAGGCTACTAG-3′ (SEQ ID NO: 10), 20 ng of genomic T7 DNA, and 2.5 U of Taq DNA polymerase (Promega) is placed in a Perking Elmer thermal cycler well. After 5 min at 95° C., the thermal cycler performs 25 cycles of the following steps: 1 min at 95° C., 1 min at 55° C., 2.5 min at 72° C. Prior to restriction the amplified DNA is purified with a DNA clean-up kit (Qiagen). The PCR products are restricted with the enzymes EcoRI and HindIII and inserted into EcoRI/HindIII cut pUC18.

Selection of T7 DNA Polymerases with Low Fidelity

For selection of T7 DNA polymerases with low fidelity, the pUC18 derivatives encoding the variants are transformed into E. coli JM101 harboring the genetic element with the inactivated tetracycline efflux pump (see Example 1). The cells are allowed to grow in LB medium containing 200 μM IPTG to induce T7 DNA polymerase gene expression, and ampicillin and chloramphenicol as selection markers for the plasmid and the genetic element. In addition, 5 mg per liter tetracycline is added to select for reconstitution of the inactivated efflux pump. After reaching a cell density of about 1 g CDW per liter, the cells are plated out on LB agar plates containing ampicillin and tetracycline. Single colonies are selected and transferred to 5 ml LB containing ampicillin in a concentration of 150 mg per liter. After incubation over night at 37° C., the pUC18 derivatives encoding the T7 DNA polymerase variants are isolated using a small-scale plasmid purification kit (Qiagen).

### Characterization of T7 DNA Polymerase Variants

The pUC18 derivatives containing the T7 DNA polymerase variants are sequenced using the M13/pUC-40 primers (MWG-Biotech). Plasmids harboring genes that contain mutations leading to amino acid substitutions in the polymerase are transformed into E. coli BH 215. Expression of the genes and purification of the T7 DNA polymerase variants is done as described earlier (Slaby, I., and Holmgren, A., Protein Expr. Purif. 2:270-277, 1991). Subsequently, the fidelity of the DNA polymerases is determined.

### Example 3

### Directed Evolution of Triosephosphate Isomerase (TIM) from a Psychrophilic Bacterium

This example illustrates the engineering of a metabolic enzyme by continuous evolution with the method shown in Example 1. The triosephosphate isomerase (TIM) from Vibrio marinus (Alvarez, M., et al., J. Biol. Chem. 273:2199-2206, 1998) has been chosen as a model enzyme.

Increasing the Thermostability of Tim from Vibrio Marinus

The independently reproducing genetic element and the host strain for in vivo mutagenesis are constructed as described in Example 1. In addition, an error-prone bacteriophage T7 DNA polymerase placed under the control of the tac promoter is introduced into the triosephosphate isomerase gene (tim) of the host chromosome by using the pKO3 system (Link et al., supra). Subsequently, the independently mutagenizing vector encoding the TIM from Vibrio marinus is inserted into this knockout strain. This strain is grown overnight in LB at 15° C. in the presence of 200 μM IPTG to induce the production of the bacteriophage T7 DNA polymerase. The culture is then diluted 1:100 into M63 medium, supplemented with 200 μM IPTG, 0.2% (v/v) glycerol as carbon source, and allowed to grow at 30° C. Under these conditions, cells synthesizing a TIM variant with improved thermostability are enabled to divide faster than the ones synthesizing the wild-type protein. This results in an enrichment of improved TIM proteins, and further modification and potential improvement.

Selection of the most thermostable TIM is done in a continuous culture. To this end, the culture is transferred into a bioreactor containing M63 medium with 0.2% (v/v) glycerol as sole carbon source. The cells are allowed to grow at 30° C. with a continuously increasing dilution rate. When wash-out starts, the cultivation is stopped and samples are plated out on LB agar plates. The TIM proteins from single colonies are characterized and the corresponding genes are sequenced.

### Example 4

### Protein Library Generation with Elements from Saccharomyces Ty5 Virus

This example illustrates the construction of an independently reproducing element derived from a reverse transcribing RNA virus. Saccharomyces Ty5-6p virus has a genome of about 5370 bp and encodes between its long terminal repeats (LTR) homologues of retroviral gag and pol genes. In contrast to retroviruses, it does not contain the env genes that are responsible for forming the viral envelope and for allowing the particle to exit the cell.

### Construction of the Host Strain

For the development of a system based on the Saccharomyces Ty5-6p virus, a Saccharomyces paradoxus minilibrary is constructed and pBluescript vectors containing the Ty5 are isolated as described by Zou et al. (Zou, S., et al., PNAS 92:920-924, 1995). From this plasmid, the gag and pol genes are amplified by PCR, concomitantly introducing Sall and BamHI restriction sites. Next, the PCR product is introduced into the expression vector YEp51, which puts the genes under control of the GAL promoter. In addition, an upstream activating sequence (UAS), e.g. a CT-box, is introduced upstream of the promoter. The UAS, the promoter, and the gag and pol genes are subsequently introduced into the ura gene of Saccharomyces cerevisiae BY4714 (ATCC 200877) using a pRS derivative (Baker Bachmann, C., et al., Yeast 14:115-132, 1998). The resulting strain is named Saccharomyces cerevisiae BY4714dU.

### Construction of the Genetic Element

Plasmid pNK254 (Zou, S., et al., PNAS 94:7412-7416) serves as source for the construction of the mutagenizing vector. The gag and pol genes are eliminated from this vector and the gene of interest is introduced between the LTR sequences under control of the GAL promoter. The intron containing his3 gene remains on the plasmid for selection of reverse transcribed genes of interest. The constructed pNK254 derivative harboring the gene of interest is introduced in Saccharomyces cerevisiae BY4714dU. Subsequently, the cells are allowed to grow in synthetic complete media without uracil and with galactose at 23° C. for 2 days. Afterwards the cultures are centrifuged, the supernatant discarded, and the cells washed twice with 100 mM MES buffer pH 6. After centrifugation, the cells are transferred to synthetic complete media without histidine, and allowed to grow for additional 2 days at 23° C. This step selects for cells containing a mutagenizing vector that has undergone a reverse transcription step. Galactose serves as a carbon source, which also induces the expression of the gene of interest. After screening and selection of cells harboring the protein with the desired properties, the corresponding gene is isolated by PCR using primers that bind at the LTR sequences of TY5. In doing so, variant genes that are on the pNK254 derivate or that are integrated into the host chromosome can be isolated. Finally, the variant genes are characterized and the proteins synthesized by using an expression system of choice in suitable host.

### Example 5

### Directed Evolution of the Tem-1β-Lactamase

This example illustrates the directed evolution of the TEM-1β-lactamase using the mutagenizing vector from Example 1, chromosomally encoded error-prone T7 DNA polymerase, and a helper phage that provides auxiliary proteins.

Cloning of the TEM-1β-Lactamase into the Mutagenizing Vector

The TEM-1β-lactamase is amplified from pUC18 (Yanish-Perron et al., supra) using the primers 5′-CTACGGGGTCTGAAGCTTAGTGGAACG-3′ (SEQ ID NO: 23) and 5′-CTGCTCCCGTGATCAGCTTACAGACAAG-3′ (SEQ ID NO: 24), which introduce a BclI site upstream and a HindIII site downstream of the bla gene. The PCR product is subsequently digested with the restriction enzymes BclI and HindIII, and ligated into BamHI/HindIII cut mutagenizing vector containing the T7 origin of replication from Example 1 that still harbors the repA gene.

### Construction of the Host Strain

An error-prone T7 DNA polymerase (e.g. from Example 2) is introduced into the chromosome of E. coli B by the method of Link et al. (supra).

### Directed Evolution of the TEM-1β-Lactamase

The mutagenesis plasmid harboring the gene coding for the wild-type TEM-1β-lactamase is introduced into the constructed host strain by electroporation. A 5 ml culture of the strain is grown at 37° C. in LB with 30 μg ml⁻¹ chloramphenicol and 200 μM IPTG to an OD₆₀₀ of 1. Then, 10⁵ T7 phage particles carrying an amber mutation in the T7 DNA polymerase gene are added and the culture is incubated at 37° C. until complete lysis is achieved. The cell debris are subsequently removed by centrifugation and 3 ml of the supernatant is mixed with 6 ml of 200 mM NaOH, 1% (w/v) SDS in water. After the addition of 4.5 ml of a solution that is 3 M with respect to sodium and 5 M with respect to acetate (pH 4.8), the mixture is incubated on ice for 10 min. The formed solids are removed by centrifugation for 20 min at 15'000×g and the supernatant transferred to a Sorvall SS34 tube containing 8.4 ml isopropanol. The tube is incubated at −20° C. for 20 min and the precipitated plasmid DNA recovered by centrifugation at 30'000×g. The supernatant is discarded and the pellet washed twice with 70% ethanol (v/v). Subsequently, the pellet is dried by aspiration and by incubation at room temperature for 20 min. Afterwards, the plasmid DNA is resuspended in 50 μl water and electroporated into E. coli B. The cells are plated out on LB plates containing 0.01, 0.02, 0.03, 0.04, and 0.05 μg ml⁻¹ cefotaxime. Cells are selected from the plate with the highest cefotaxime concentration that has any colonies. From this point, either the plasmid is isolated and the DNA sequence of the β-lactamase gene is determined, or a 5 ml culture for an additional infection and mutagenesis round is prepared. In the latter case, selection is performed at cefotaxime concentrations that are higher than the one on which the cells were selected in the previous round. The cycling is repeated until an enzyme with the desired activity could be selected. Finally, the corresponding gene is isolated and sequenced, and the protein characterized.

### Example 6

### Engineering of Green Fluorescent Protein (GFP) for Different Emission Wavelengths

This example illustrates the engineering of green fluorescent protein (GFP) of the jellyfish Aequorea victoria for longer wavelength emissions than wild-type enzyme by using the mutagenizing vector described in Example 1. Furthermore, it shows a possible screening method for use in combination with the presented invention. The generated GFP-variants provide distinguishable markers to monitor e.g. multiple cellular events simultaneously.

Cloning of the GFP Gene into the Mutagenizing Vector

The GFP gene of Aequorea victoria including a heterologous promoter is amplified by PCR from an existing GFP vector and cloned into the Pstl site of pUC18 by methods well known in the art. From this vector, the gene is excised as a BamHI/HindIII fragment and introduced into the mutagenizing vector from Example 1 that still encodes the repA protein.

### Directed Evolution of GFP

The mutagenizing vector harboring the GFP gene is co-transformed with a pUC18 plasmid encoding an error-prone T7 DNA polymerase into the host strain described in Example 1. The cells are allowed to grow at 37° C. on LB medium containing 200 μM IPTG for recombinant gene expression, and ampicillin and chloramphenicol as selection markers for the pUC18 derivative and the mutagenizing vector, respectively. At an OD₆₀₀ of 2, the cells are plated on LB agar plates containing ampicillin and chloramphenicol, and the plates are incubated overnight at 30° C. The obtained colonies on agar plates are visually screened for different emission colors and ratios of brightness when excited at 475 vs. 395 nm, supplied by a xenon lamp and grating monochromator for which the output beam is expanded to illuminate an entire culture dish. Selected colonies are subsequently purified on LB agar plates containing chloramphenicol. For further characterization, 5 ml cultures of the obtained strains are grown to an OD₆₀₀ of 2 on LB, 200 μM IPTG, and chloramphenicol. 1.5 ml of this culture are transferred to an Eppendorf tube, centrifuged, washed, and resuspended in 150 μl of 50 mM Tris-HCl (pH 8)/2 mM EDTA. Lysozyme and DNasel are then added to 0.2 mg ml⁻¹ and 20 μg ml⁻¹, respectively, and the samples are incubated on ice for 2 hours. Afterwards, cleared cell extracts are prepared by centrifugation at 12'000×g for 15 min. and the supernatants analyzed by fluorescence spectroscopy. Next, the GFP variants with the desired properties are selected and the genes are isolated from the corresponding master plates. They can subsequently be used for different applications such as for the construction of new expression vectors.

1. A method for the in vivo generation of a library of variants of polynucleotides comprising culturing a host cell wherein the host cell
i) contains a genetic element harboring a viral or phage origin of replication,
ii) harbors a viral or phage error-prone polymerase that is involved in replication of said genetic element (i), but which is not physically linked to said genetic element (i),
iii) harbors viral or phage auxiliary nucleotide sequences and proteins that are required for replication of said genetic element (i),
iv) contains a nucleotide sequence or several nucleotide sequences of interest that are physically linked to said viral or phage origin of replication (i),
v) replicates its genome independently of said genetic element (i).

2. The method of claim 1 wherein the genetic element harbors a phage origin of replication.

3. The method of claim 1 wherein the genetic element harbors a T7 phage origin of replication.

4. The method of claim 3 wherein the phage error-prone polymerase and the phage auxiliary nucleotide sequences and proteins are from phage T7.

5. The method of claim 1 wherein the error-prone polymerase is encoded on a chromosome, plasmid, cosmid, or artificial chromosome in the host cell.

6. The method of claim 1 wherein the error-prone polymerase is provided by a virus or phage.

7. The method of claim 1 wherein the auxiliary proteins are encoded on a chromosome, plasmid, cosmid, or artificial chromosome in the host cell.

8. The method of claim 1 wherein the auxiliary proteins are provided by a virus or phage.

9. A method for the generation of polynucleotides with desired properties or polynucleotides encoding peptides or proteins with desired properties, wherein
i) a library of nucleotide sequence variants is constructed by culturing a host cell as claimed in claim 1,
ii) said library (i) is screened and selected for host cells producing variants with desired properties,
iii) said selected host cells (ii) are isolated,
iv) the variant nucleotide sequences of interest on the genetic elements of said isolated host cells (iii) are isolated and characterized.

10. The method of claim 9 wherein in step ii) the polynucleotide library is expressed to give a peptide or protein library and the peptide or protein variants are screened for the desired property.

11. The method of claim 9 wherein the steps i), ii) and iii) are repeated one or more times.

12. A method of manufacture of a peptide or protein with desired properties wherein a variant nucleotide sequence with desired properties is generated and isolated according to claim 9 and said nucleotide sequence expressed in a suitable host cell.

13. Use of a peptide or protein manufactured according to claim 12 as a therapeutic, catalyst, detergent, cosmetic, or feed additive.

14. A kit comprising at least two out of the three components being
i) a genetic element harboring a viral or phage origin of replication, into which a nucleotide sequence or several nucleotide sequences of interest can be introduced,
ii) a nucleotide sequence encoding a viral or phage error-prone polymerase that is involved in replication of said genetic element, and
iii) one or several nucleotide sequences coding for auxiliary proteins that are required for replication of said genetic element (i).
